(19) United States
(12) Patent Application Publication
(10) Pub. No.: US 2012/0197075 A1
(43) Pub. Date: Aug. 2, 2012

(54) ENDOSCOPE SECUREMENT DEVICE AND METHOD OF USE

(76) Inventors: William Krimsky, Owings Mills, MD (US); Gregory R. McArthur, Sandy, UT (US)

(21) Appl. No.: 13/285,152

(22) Filed: Oct. 31, 2011

Related U.S. Application Data
(60) Provisional application No. 61/408,946, filed on Nov. 1, 2010.

(51) Int. Cl. A61B 1/00

(52) U.S. Cl. 600/102

(57) ABSTRACT

An endoscope securement device for coupling an endoscope to the body of a practitioner. The endoscope securement device includes a base capable of being coupled to a practitioner and an articulable arm extending from the base configured to be coupled to an endoscope. The device allows a practitioner to manipulate the endoscope through his or her body movements, leaving his or her hands free to manipulate accessories to the device or other tools.

### CROSS REFERENCE TO RELATED APPLICATIONS

The present application claims priority to U.S. Provisional Application No. 61/408,946, filed on Nov. 1, 2010, and entitled “ENDOSCOPE SECUREMENT DEVICE AND METHOD OF USE,” which is hereby incorporated by reference in its entirety.

### TECHNICAL FIELD

The present disclosure relates generally to medical instrument securement devices. More specifically, the present disclosure relates to devices for securing an endoscope to a physician in order to facilitate use of the endoscope.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an endoscope securement device;

FIG. 2 is a perspective view of the endoscope securement device of FIG. 1 coupled with an endoscope;

FIG. 3 is a partially cutaway perspective view of an endoscope securement device configured in a curved position;

FIG. 4 is an exploded perspective view of a portion of the endoscope securement device of FIG. 1;

FIGS. 5A, 5B, and 5C are cross-sectional views of arm segments of an endoscope securement device;

FIG. 6A is an exploded perspective view of a locking mechanism for an endoscope securement device;

FIG. 6B is a side elevation view of a rotatable body component of the locking mechanism of FIG. 6A;

FIG. 7 is a view of an endoscope securement device in use, coupled to a physician.

### DETAILED DESCRIPTION

An endoscope securement device may be utilized by a physician during treatment of a patient. An endoscope securement device that couples the endoscope to the body of the physician may allow the physician to adjust the position of the endoscope without the use of his or her hands. This in turn allows the physician to position the endoscope while simultaneously manipulating or controlling other devices or instruments.

It will be readily understood that the components of the embodiments as generally described and illustrated in the Figures herein could be arranged and designed in a wide variety of different configurations. Thus, the following more detailed description of various embodiments, as represented in the Figures, is not intended to limit the scope of the disclosure, but is merely representative of various embodiments. While the various aspects of the embodiments are presented in drawings, the drawings are not necessarily drawn to scale unless specifically indicated.

The phrases “connected to,” “coupled to,” and “in communication with” refer to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

The term “articulate,” as used herein, refers to manipulating or adjusting a jointed member into alternate configurations or shapes. Thus an “articulable” member is a jointed member capable of being adjusted into alternate configurations. In the case of an elongate articulable member, the member could be manipulated, for example, to be substantially straight, or to be curved in any variety of directions, or straight for portions of its length and curved along other portions.

The terms “vertical” and “horizontal” as used herein are given their standard meanings. When referring to a “horizontal” or “vertical” surface or edge of the endoscope securement device described below, the endoscope securement device is oriented as it would be oriented while in use. Further the “proximal” and “distal” ends of the endoscope securement device described are the ends near to, and distant from (respectively), a practitioner using the endoscope securement device.

The term “axial” is also given its ordinary meaning. Thus, the “axial direction” is a direction along the axis of a part. As used below, the axis of substantially cylindrical parts of the endoscope securement device refers to the center axis of the cylindrical portion of the part. The axial direction of a cord is a direction along the length of the cord. The axial direction need not be in a straight or substantially straight line, thus the axial direction of a curved tubular component would follow the curvature of the component.

The disclosure below refers specifically to an endoscope securement device. As will be appreciated by those skilled in the art, the disclosure is not limited in its application to endoscopes. Rather, a variety of medical devices and tools can be secured in a like manner by the device disclosed below.

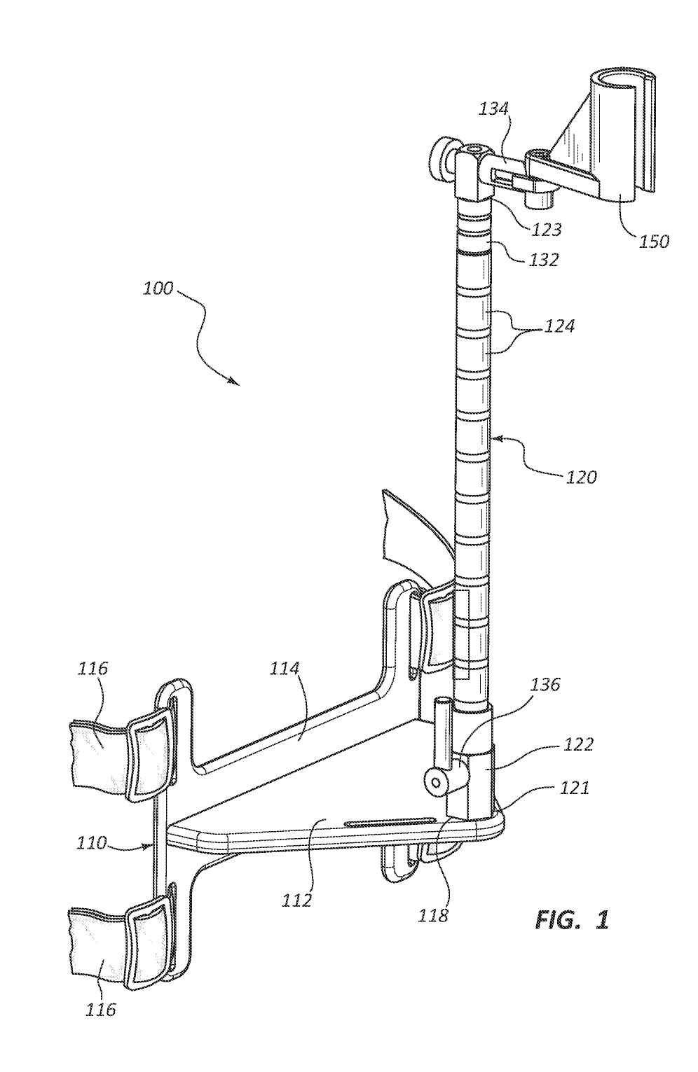

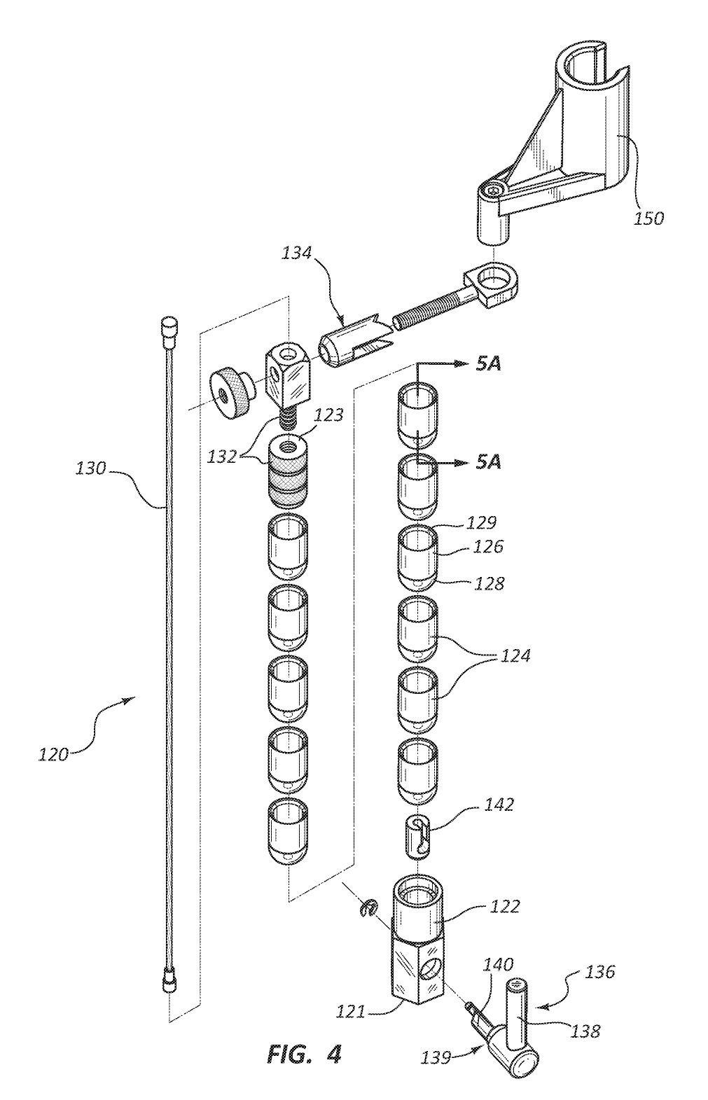

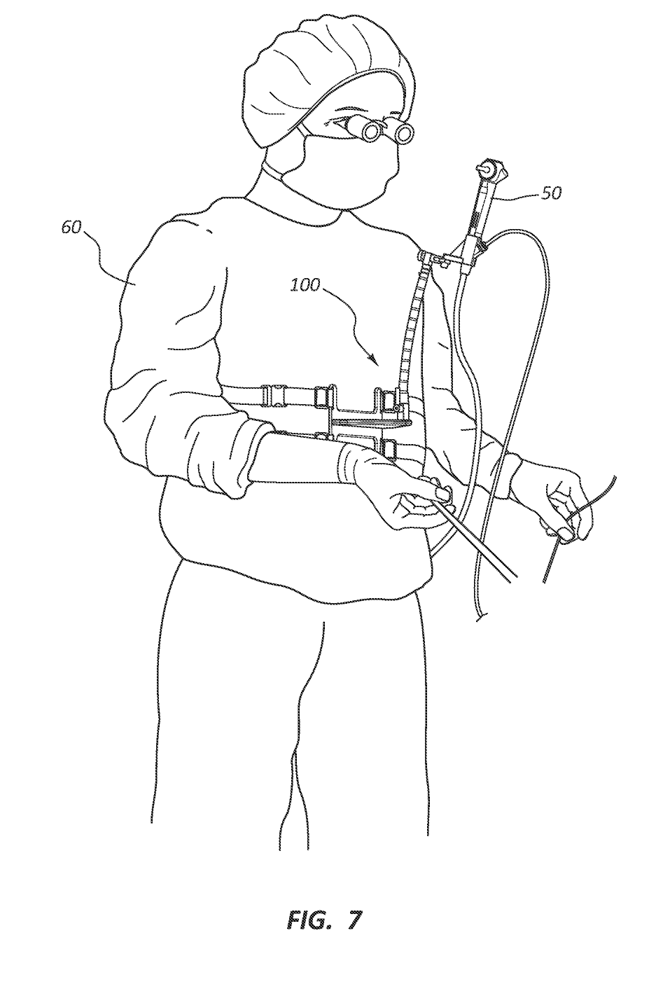

FIGS. 1-3 show assembled views of an endoscope securement device. FIGS. 4-6B show details of particular components an endoscope securement device. FIG. 7 shows an endoscope securement device secured to a physician.

Referring generally to FIGS. 1-3, an endoscope securement device 100 may include a base 110, an articulable arm 120, and an endoscope dock 150.

In one embodiment, the base 110 has a first surface 112 and a second surface 114. When the securement device is in use, the first surface 112 is disposed in a substantially horizontal position and the second surface 114 in a substantially vertical direction. The first surface 112 and the second surface 114 may include two separate pieces of material, coupled together in a substantially perpendicular configuration, or the two surfaces may be two surfaces of a single, integrally formed part.

Straps 116 may be coupled to the base 110 to provide a means whereby the endoscope securement device can be attached to a practitioner. When in use, the second surface 114 of the base 110 may be disposed adjacent to the front-midsection of a practitioner. Straps 116 may then extend around the body of the practitioner, thereby coupling the device to the body of the practitioner. As will be appreciated by one skilled in the art, the endoscope securement device 100 could alternatively be coupled to a variety of positions on a practitioner. For example, as an alternative to being coupled to the front midsection of a practitioner, the endoscope securement device 100 could be coupled to the shoulder or hip of a practitioner, or any other comfortable location. Furthermore, a variety of configurations of straps or similar securement mechanisms can be utilized to couple the endoscope securement device to a practitioner. For example, two straps 116 may be coupled to the second surface 114 of the base 110. The straps 116 may be equipped with buckles and tightening mechanisms known in the art to facilitate the use of the straps 116. Other like attachment mechanisms such as ties, cords, belts, or webbing may similarly be used.

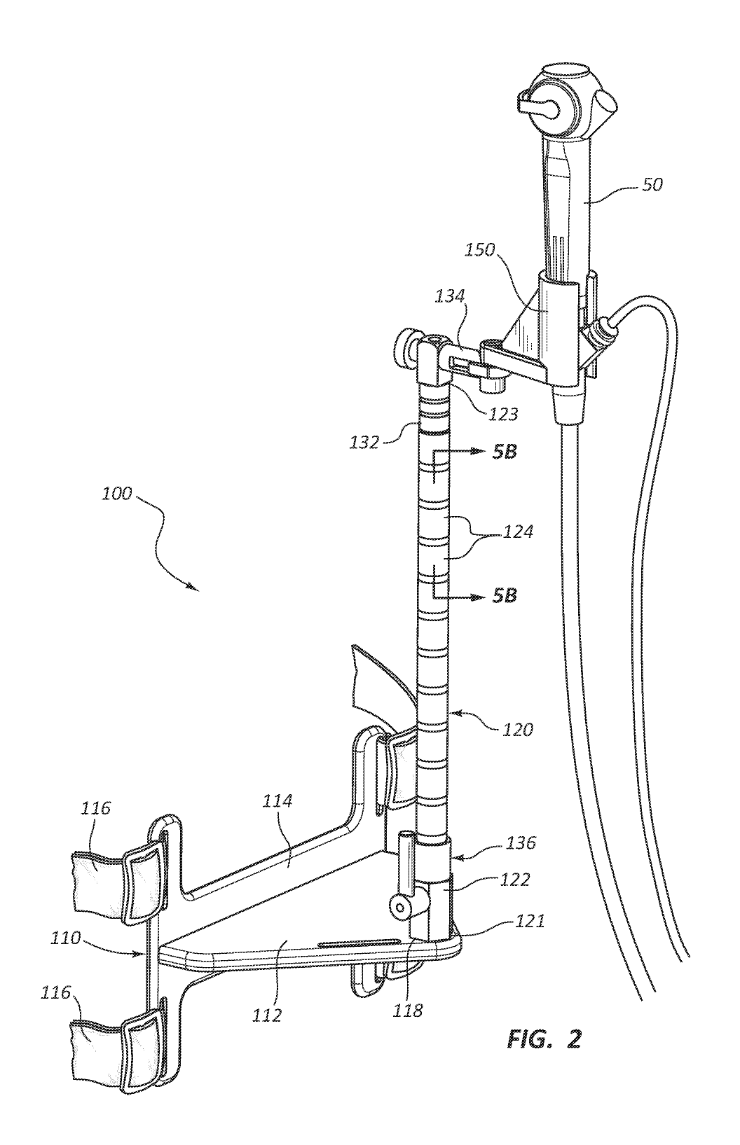

As mentioned, in one embodiment, two straps 116 are coupled to the second surface 114 of the base. The vertical displacement of the ends of the first strap with respect to the second strap may impact the stability with which the device is attached to a practitioner. Likewise, the horizontal displacement of a first end of one strap with respect to a second end of the same strap may impact the stability with which the device is attached to a practitioner. Thus, in certain embodiments, the straps will be attached near the top and bottom of the vertical edges of the second surface and adjacent to the vertical edges of the second surface as depicted in FIGS. 1 and 2.

Referring to FIGS. 1 and 2 generally, it will be further appreciated by those skilled in the art that the straps 116 may be attached to the base 110 in a variety of configurations and arrangements. For example, the straps 116 may be coupled to the second surface 114 near the vertical edges as described above, or offset from the vertical edges. Likewise the straps 116 need not be adjacent to the top and bottom of the vertical edges, but may be offset. Moreover, straps 116 may alternatively be attached to the first surface 112 of the base 110.

Though a configuration of two straps is described above, the securement device may employ fewer straps, or more straps, in an analogous manner. As will be appreciated by one skilled in the art, a variety of configurations of straps, disposed in a variety of positions on the base 110, may be used to attach the securement device to a practitioner.

Referring again to FIGS. 1-3 generally, the first surface 112 of the base 110 may be disposed in a substantially horizontal position when the endoscope securement device 100 is in use. The first surface 112 may further provide a mounting point 118 for the articulable arm 120 of the endoscope securement device 100. The distance along the first surface 112 between the mounting point 118 and the second surface 114 may vary in different embodiments of the device. Further, the mounting point 118 may be located at any position on the first surface 112 of the securement device.

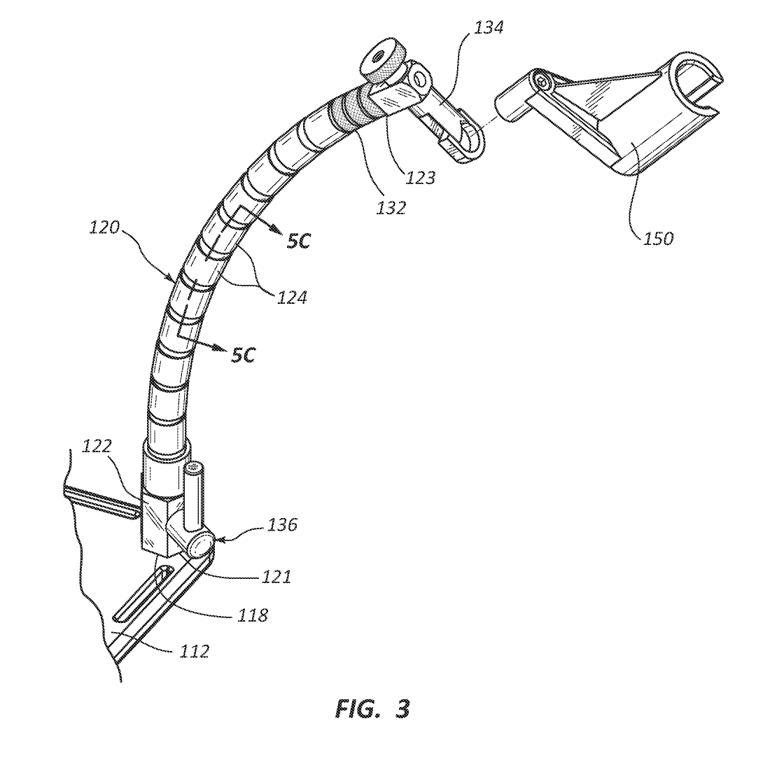

An articulable arm 120 may be coupled to the base 110 at the arm mounting point 118. The articulable arm 120 may have a proximal end 121 and a distal end 123. As used herein, an end of the articulable arm 120 of the endoscope securement device which is coupled to the base 110 of the endoscope securement device is the proximal end of the articulable arm 120 and the opposite end is the distal end of the articulable arm 120. The articulable arm 120 may have an arm attachment member 122 at the proximal end 121 of the articulable arm 120. The arm may further include a plurality of arm segments 124, each having a substantially cylindrical shape. In other embodiments, the arm segments may have a geometry other than cylindrical. In one embodiment these arm segments 124 are aligned axially to form a portion of the length of the arm. As described in further detail below, these arm segments 124 may be allowed to move with respect to each other, thereby allowing the articulable arm 120 to be manipulated into a variety of shapes and positions. For example, FIGS. 1 and 2 depict an articulable arm 120 in a substantially straight position while FIG. 3 depicts an articulable arm 120 in a curved position.

Continuing to refer to FIGS. 1-3 generally, a locking mechanism 136 may be provided in connection with the articulable arm 120. In one embodiment the locking mechanism 136 allows a practitioner to unlock the arm, thereby allowing the arm segments 124 to move with respect to each other. In the unlocked position, the practitioner may then manipulate the articulable arm 120 into a desired position. The practitioner may then utilize locking mechanism 136 to lock the articulable arm 120 in the desired position, preventing movement of the arm segments 124 with respect to each other. Further features of the articulable arm 120 and locking mechanism 136 are described in conjunction with other Figures below.

The endoscope securement device 100 may also include an endoscope dock 150. In one embodiment, the endoscope dock is secured to the articulable arm 120 by a clamp 134 adjacent the distal end of the articulable arm 120. The endoscope dock 150 may be configured to hold a variety of shapes and types of endoscopes. Further, the clamp 134 may be configured to allow rotation of a portion of the clamp 134 and the endoscope dock 150 about the axis of the articulable arm 120 and about an axis transverse to the axis of the articulable arm 120.

FIG. 2 depicts one embodiment of an endoscope 50, placed in the endoscope dock 150. As will be appreciated by those skilled in the art, the endoscope dock 150 may be formed from a variety of materials such as metal or plastic. Further, the endoscope dock 150 may be a single integrally formed part or a plurality of smaller parts coupled together.

The endoscope dock 150 may be configured to hold an endoscope by friction and gravity or the device may further include additional mechanisms to couple the endoscope to the endoscope dock 150, such as a clamp.

Referring now to FIG. 4, FIG. 4 depicts an exploded view of one embodiment of an articulable arm 120. As previously described, the articulable arm 120 may include an arm attachment member 122 and a plurality of arm segments 124. The articulable arm 120 may further include an adjustment mechanism 132 and a locking mechanism 136.

The arm attachment member 122, the plurality of arm segments 124, and the adjustment mechanism 132 may be substantially cylindrical. In one embodiment, the articulable arm 120 is formed by axially aligning these substantially cylindrical parts. In this embodiment the cylindrical parts are configured with a hole along the axis of each part. In some embodiments the cylindrical part and the axial hole are configured such that the part is substantially hollow. Axially aligning the substantially cylindrical parts in this way creates an articulable arm 120 assembly with a more or less tubular geometry. As used herein, “substantially cylindrical parts” refers to parts that are substantially cylindrical as depicted in the Figures. It is not necessary for the functionality of the device that these parts actually be substantially cylindrical. It will be appreciated by those skilled in the art that other shapes may also be employed.

When the substantially cylindrical parts are aligned, the parts may also be threaded onto a cable 130. In one embodiment, the cable 130 is fixed to the parts located at the proximal 121 and distal 123 ends of the articulable arm 120. The remaining parts are threaded on the cable 130 but allowed to rotate freely and, to the extent the cable 130 is longer than the length of the “tube” or “sheath” formed by the parts, the remaining parts are free to translate along the axis of the cable 130.

The cable 130 may further be configured to provide a compressive axial force on the substantially cylindrical parts. This force is applied when one or both ends of the cable 130 move in such a manner as to create tension in the cable 130, thereby causing the cable 130 to exert a compressive force on the “sheath” or “tube” formed by the substantially cylindrical parts. “Tightening” of the cable 130 causes the cable 130 to exert an axial compressive force on the substantially cylindrical parts because “tightening” the cable draws the parts at the proximal 121 and distal 123 ends of the articulable arm 120 closer together along the axis of the articulable arm 120. This “tightening” of the cable 130 may be accomplished by a locking mechanism 136, or the adjustment mechanism 132, or both. Likewise, the cable 130 may be loosened (or configured such that it is not applying a compressive axial force on the cylindrical parts) by the locking mechanism 136, or the adjustment mechanism 132, or both.

When the cable 130 applies a compressive axial force on the cylindrical parts, friction between the cylindrical parts may prevent movement of the parts relative to each other. When the cable 130 is loosened, the parts may move in relation to each other, constrained by the cable 130 running through the hole in each cylindrical part.

In one embodiment the cable 130 is coupled to a portion of the clamp 134 adjacent the distal end of the cable 130. The cable 130 extends through the adjustment mechanism 132 and the plurality of arm segments 124. The cable is then coupled to a cable holder 142 adjacent the proximal end of the cable 130. As described in more detail below, in one embodiment, the cable holder 142 is part of the locking mechanism 136. The locking mechanism may be configured to alternate between “tightening” the cable relative to the cylindrical parts, thus applying a compressive axial force to the cylindrical parts, and “loosening” the cable 130 relative to the cylindrical parts, thus removing the compressive axial force.

As described above, an adjustment mechanism 132 may be provided in connection with the articulable arm 120. The adjustment mechanism 132 may be composed of two substantially cylindrical parts, configured to allow the cable 130 to pass through each part along its axis. The two components of the adjustment mechanism may move relative to each other in the axial direction and overlap in the axial direction. When the components are moved relative to each other such that there is less overlap between the two components, the adjustment mechanism 132 is effectively “lengthened” in the axial direction. In one embodiment the adjustment mechanism is composed of two cylindrical parts having mating threads. When the parts are rotated with respect to each other, the mating threads will cause the parts to translate in the axial direction. In one embodiment, “lengthening” or “shortening” the adjustment mechanism 132 in turn adds or removes length from the “tube” or “sheath” formed by the substantially cylindrical parts.

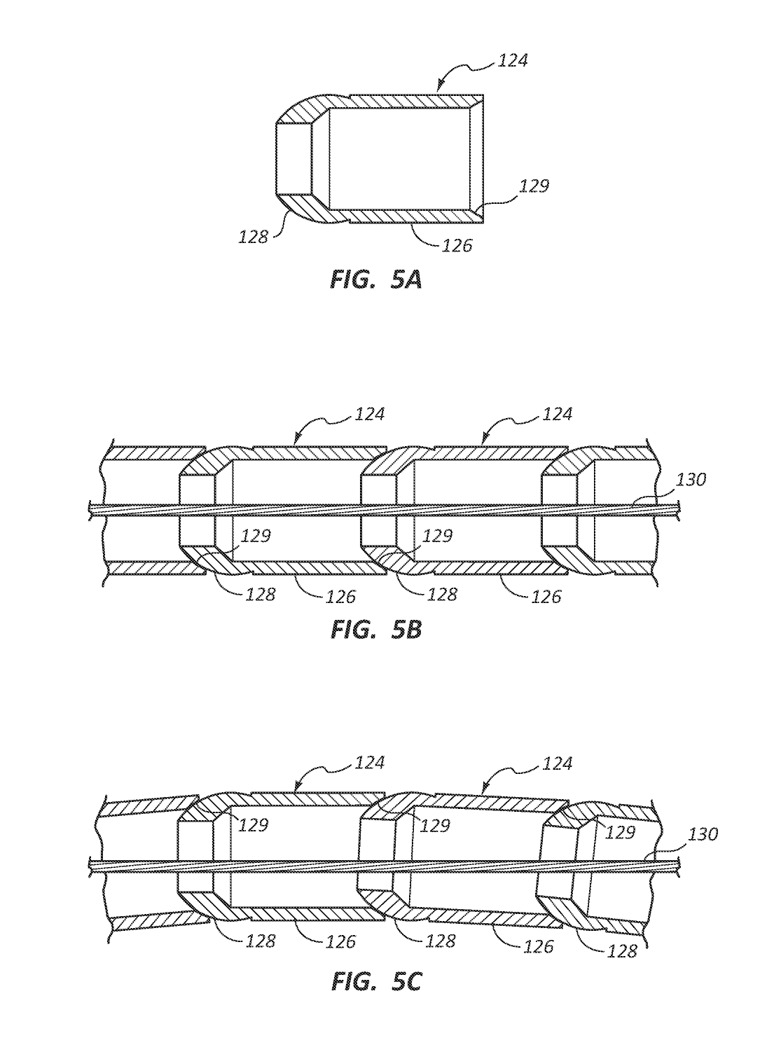

FIG. 5A is a cross sectional view of an individual arm segment 124. FIGS. 5B and 5C are cross sectional views of a portion of the articulable arm 120 depicting how the arm segments 124 interface. Referring to these Figures generally, in one embodiment, each arm segment has a substantially cylindrical body portion 126 and a hemispherical top portion 128. The bottom of each cylindrical body portion 126 may further include a seat 129. The seat 129 may be configured to contact the hemispherical top portion 128 of a mating arm segment 124. Thus, referring again to the exploded view in FIG. 4, in one embodiment, when the arm segments 124 are assembled, each hemispherical top portion 128 will be in contact with the seat 129 of an adjacent arm segment 124. Parts axially aligned with the arm segments 124 at the proximal 121 and distal 123 ends of the articulable arm 120, such as the arm attachment member 122 and the adjustment mechanism 132, may also be configured to mate with either the seat 129 or the hemispherical top portion 128 of an adjacent arm segment 124.

In one embodiment, when an axial compressive force is applied to the arm segments 124, the contact force between the hemispherical top portion 128 and the seat 129 of adjacent arm segments 124 increases. This increase in contact force necessarily increases the force of friction between adjacent arm segments. Thus, when an axial compressive force is applied, the force of friction between the hemispherical top portion 128 and the seat 129 of adjacent arm segments 124 restricts movement of the adjacent arm segments 124 relative to each other.

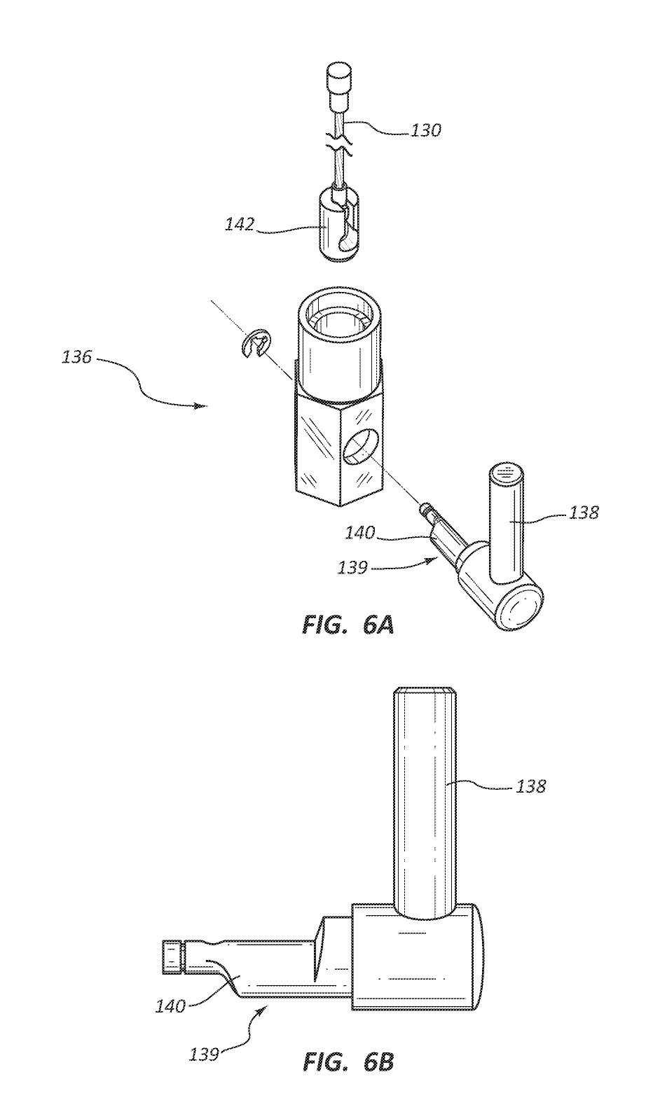

FIG. 6 depicts a detailed view of a locking mechanism 136. In the illustrated embodiment, the locking mechanism 136 includes a lever 138, a rotating body member 139, a cam 140, and a cable holder 142. The rotating body member 139 passes through the arm attachment member 122 and the cable holder 142. The cable holder 142 is coupled to the cable 130. As the rotating body member 139 rotates, the cam 140 engages the cable holder 142, causing it to translate in the axial direction of the articulable arm 120. Depending on the rotation of the cam 140, this translation either tightens or loosens the cable 130.

FIG. 7 depicts an endoscope securement device 100 in use, attached to a physician 60. When the endoscope securement device 100 is coupled to the midsection of a physician, the physician 60 is able to manipulate the endoscope or adjust its position using body movements, thus leaving one or both of his or her hands free to use other instruments. For example, a physician performing surgery through an endoscope may need to simultaneously adjust the position of the endoscope while also manipulating tools inserted through lumens of the endoscope. The physician could manipulate the endoscope by moving his or her body while his or her feet remain stationary, or by twisting his or her body, or by walking toward or away from the patient, or a combination of these and other body movements.

Similarly, a physician may initially use his or her hands to introduce an endoscope into a patient and position the endoscope at a desired point. Through use of the endoscope securement device disclosed herein, a physician could then make minor adjustments during the therapeutic procedure to the position of the endoscope through his or her body movements alone, or in combination with his or her hands.

In another instance, the physician may use the articulable arm 120 of one of the embodiments of endoscope securement described herein to further control the position of the endoscope. For example, the physician may initially position the endoscope using his or her hands. The physician could then manipulate the articulable arm such that the endoscope remains in the desired position while also allowing the physician to position him or herself in a comfortable and proper location to perform the necessary therapy. During the procedure, the physician could adjust the position of the endoscope with body movements alone or could adjust the position of the endoscope using a combination of adjustments to his or her body position and the position of the articulable arm. The numerous potential positions of the articulable arm in combination with the numerous body movements a physician may utilize to manipulate an endoscope coupled to the securement device, result in a versatility that may be conceptualized as creating a “third arm” for the physician during surgery.

While specific embodiments of endoscope securement devices have been illustrated and described, it is to be understood that the disclosure provided is not limited to the precise configuration and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

1. An endoscope securement device comprising:
a base configured to be coupled to a user;
an articulable arm, configured to be releaseably locked in multiple configurations, the articulable arm coupled to the base, and the articulable arm comprising a plurality of segments;
an endoscope dock coupled to the articulable arm, the endoscope dock configured to receive an endoscope.

2. The endoscope securement device of claim 1, further comprising a locking mechanism configured to toggle the articulable arm in a locked and an unlocked position.

3. The endoscope securement device of claim 2, wherein the locking mechanism comprises a rotatable body member and a cam, configured to translate a first end of a cable disposed along the axis of the articulable arm, the cable configured to selectively apply an axial compressive force on the plurality of arm segments.

4. The endoscope securement device of claim 1, further comprising a cable disposed along an axis of the articulable arm, the cable configured to selectively apply an axial compressive force on the plurality of arm segments.

5. The endoscope securement device of claim 1, wherein the endoscope dock is coupled to the articulable arm by a clamp.

6. A method of manipulating an endoscope comprising:
coupling an endoscope securement device to the midsection of a user, the endoscope securement device comprising:
a base configured to be coupled to a user;
an articulable arm, configured to be releaseably locked in multiple configurations, the articulable arm coupled to the base, and the articulable arm comprising a plurality of segments;
an endoscope dock coupled to the articulable arm, the endoscope dock configured to receive an endoscope;
manipulating the endoscope through at least one hands-free movement of the user.
